(19) United States
(12) Patent Application Publication
(10) Pub. No.: US 2006/0263400 A1
(43) Pub. Date: Nov. 23, 2006

(54) Regulation of mammalian keratinous tissue using skin and/or hair care actives

(76) Inventor: Donald Lynn Bissett, Hamilton, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/345,589

(22) Filed: Feb. 1, 2006

Related U.S. Application Data
(60) Provisional application No. 60/681,626, filed on May 17, 2005.

(51) Int. Cl. A61K 31/49; A61K 31/36

(52) U.S. Cl. 424/401

(57) ABSTRACT

Compositions useful for regulating the condition of mammalian keratinous tissue needing such treatments are provided. An exemplary composition comprises a safe and effective amount of glycyrrhizic acid and/or glycyrrhetinic acid; a safe and effective amount of an active selected from the group consisting of N-acyl amino acid compounds, hexamidine, cetyl pyridinium chloride, ergothioneine, and combinations thereof, and a dermatologically acceptable carrier.

### CROSS REFERENCE TO RELATED APPLICATION

This application claims the benefit of U.S. Provisional Patent Application Ser. No. 60/681626, filed May 17, 2005, the entirety of which is incorporated herein by reference.

### FIELD OF THE INVENTION

The present invention relates to personal care compositions containing skin and hair care actives. Such compositions are useful for regulating the condition of mammalian keratinous tissue needing such treatments, particularly skin lightening.

### BACKGROUND OF THE INVENTION

Currently, there are a number of personal care products that are available to consumers that are directed toward improving the health and physical appearance of keratinous tissue, such as the skin, hair, and nails. The majority of these products are directed to delaying, minimizing or even eliminating skin wrinkling and histological changes typically associated with the aging of skin or environmental damage to human skin. However, there also exists a need for cosmetic agents to prevent, retard, and/or treat uneven skin tone by acting as a lightening or pigmentation reduction cosmetic agent.

Mammalian keratinous tissue, particularly human skin and hair, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damage includes thinning of the skin, which occurs naturally as one ages. With such thinning, there is a reduction in the cells and blood vessels that supply the skin, as well as a flattening of the dermal-epidermal junction that results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990. Other damages or changes seen in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin (i.e., flaking, scaling, dryness, roughness). For hair, these extrinsic and intrinsic factors can contribute to, among other problems, hair bleaching, split ends, fragility, roughness, hair loss, reduction in hair growth rate, and the like. Therefore, there is a need for products and methods that seek to remedy these keratinous tissue conditions.

### SUMMARY OF THE INVENTION

The present invention is directed to topical compositions that contain certain skin and/or hair care actives, which may be used to provide prophylactic as well as therapeutic treatments for keratinous tissue conditions.

In accordance with one exemplary embodiment of the present invention, there has now been provided a personal care composition comprising a safe and effective amount of glycyrrhizic acid and/or glycyrrhetinic acid; a safe and effective amount of a first active selected from the group consisting of N-acyl amino acid compounds, hexamidine, cetyl pyridinium chloride, ergothioneine, and combinations thereof; and a dermatologically acceptable carrier.

In accordance with another exemplary embodiment of the present invention, there has now been provided a personal care composition comprising a dermatologically acceptable carrier; and a safe and effective amount of each of the following actives: glycyrrhizic acid and/or glycyrrhetinic acid, niacinamide, vitamin E or a derivative thereof, panthenol, and a sunscreen agent.

The present invention is also directed to methods of regulating the condition of mammalian keratinous tissue. In accordance with one exemplary embodiment, there has now been provided a method comprising the steps of: a) combining a first personal care composition or composition part with a second personal care composition or composition part, and b) applying the combined compositions or composition parts to the mammalian keratinous tissue, wherein step a) is conducted directly before and/or during step b). As used herein, the phrase “directly before” means within 20 seconds prior to the event or step qualified by this phrase. The first personal care composition or composition part includes a first active selected from the group consisting of glycyrrhizic acid, glycyrrhetinic acid, N-acyl amino acid compounds, ergothioneine, and combinations thereof. The second personal care composition or composition part includes a second active selected from the group consisting of hexamidine, cetyl pyridinium chloride, and combinations thereof.

### DETAILED DESCRIPTION OF THE INVENTION

All percentages and ratios used herein are by weight of the total composition and all measurements made are at 25° C., unless otherwise designated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The term “keratinous tissue,” as used herein, refers to keratin-containing layers disposed as the outermost protective covering of mammals which includes, but is not limited to, skin, hair, toenails, fingernails, cuticles, hooves, etc.

The term “topical application,” as used herein, means to apply or spread the compositions of the present invention onto the surface of the keratinous tissue.

The term “dermatologically acceptable,” as used herein, means that the compositions or components described are suitable for use in contact with human keratinous tissue without undue toxicity, incompatibility, instability, allergic response, and the like.

The phrase “safe and effective amount,” as used herein, means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

The term “post-inflammatory hyperpigmentation,” as used herein, refers to the changes in melanin content as a response to an inflammatory event (e.g., acne, scratch, insect sting or bite, sunburn, etc), especially in dark skin subjects.

The term “hyperpigmentation,” as used herein, refers to an area of skin wherein the pigmentation is greater than that of an adjacent area of skin (e.g., a pigment spot, an age spot, and the like).

The terms “desquamation, exfoliation, and/or turnover,” as used herein, mean the removal of the upper layers of the stratum corneum (comprising the horny layers).

The terms “oily and/or shiny appearance,” as used herein, mean the glossy look mammalian skin tends to exhibit upon the excretion of oil, sebum, and/or sweat from the respective source gland.

The term “sagging,” as used herein, means the laxity, slackness, or the like condition of skin that occurs as a result of loss of, damage to, alterations to, and/or abnormalities in dermal elastin.

The term “smoothing” and “softening,” as used herein, means altering the surface of the keratinous tissue such that its tactile feel is improved.

The term “sallowness,” as used herein, means the pale color, yellow color or the like condition of skin that occurs as a result of a loss of, damage to, alterations to, and/or abnormalities in skin components such that they become colored (e.g., yellow in color) due to processes such as protein glycation and accumulation of lipofuscin or in the decrease in peripheral blood flow that typically accompanies skin aging.

The compositions of the present invention are useful for topical application and for regulating keratinous tissue condition. Regulation of keratinous tissue condition, especially human skin condition, is often required due to conditions that may be induced or caused by factors internal and/or external to the body. For instance, “regulating skin condition” includes prophylactically regulating and/or therapeutically regulating skin condition, and may involve one or more of the following benefits: thickening (i.e., building the epidermis and/or dermis layers of the skin and/or the subcutaeous layers such as fat and muscle, and where applicable, the keratinous layers of the nail and hair shaft) to reduce atrophy (e.g., of the skin), increasing the convolution of the dermal-epidermal border, reducing non-melanin skin discoloration such as under eye circles, blotching (e.g., uneven red coloration due to, e.g., rosacea) (hereinafter referred to as “red blotchiness”), sallowness (pale or yellow color), reducing discoloration caused by telangiectasia or spider vessels, and reducing discolorations due to melanin (e.g., pigment spots, age spots, uneven pigmentation) and other chromophores in the skin (e.g., lipofuscin, protein crosslinks such as those that occur with glycation, and the like). As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like in the skin which may be detected visually or by feel). As used herein, therapeutically regulating skin condition includes ameliorating (e.g., diminishing, minimizing and/or effacing) discontinuities in skin. Regulating skin condition involves improving skin appearance and/or feel.

### Components

I. Glycyrrhizic Acid and/or Glycyrrhetinic Acid

Compositions of the present invention include a safe and effective amount of glycyrrhizic acid and/or glycyrrhetinic acid. Preferably, the composition contains these acid compounds in an amount from about 0.01% to about 10%, more preferably from about 0.05% to about 5%, even more preferably from about 0.1% to about 3%, by weight of the composition.

Glycyrrhizic acid is a component of licorice extract, and is an anti-inflammatory agent. Inflammatory mediators or cytokines can stimulate pigment cells (melanocytes) to produce melanin. Thus, inflammatory conditions such as UV-damage, acne, in-grown hairs, insect bites, scratches, etc. will stimulate what is called post-inflammatory hyperpigmentation. While UV is a primary inducer of pigmentation in all skin types, pigment from the other inflammatory stimuli (acne, etc.) will, in particular, contribute to skin pigmentation in darker skin individuals (e.g., Hispanic, Asian). Inhibiting inflammation with anti-inflammatory agents will reduce pigmentation. Glycyrrhizic acid is also believed to be a scavenger of nitric oxide. Nitric oxide (NO) is a stimulator of pigmentation. Use of nitric oxide scavengers (materials that react with nitric oxide to prevent it from stimulating pigment cells) will reduce pigmentation. Glycyrrhizic acid is also known as glycyrrhizin, glycyrrhizinic acid, or glycyrrhetinic acid glycoside.

Glycyrrhetinic acid is an anti-inflammatory agent. Structurally, glycyrrhetinic acid is different from glycyrrhizic acid in that glycyrrhetinic acid does not have an attached sugar residue (glycoside). Glycyrrhetinic acid is also known as enoxolone, glycyrrhetic acid, or uralenic acid.

II. Additional Actives

The personal care compositions further include a safe and effective amount of an active selected from the group consisting of N-acyl amino acid compounds, hexamidine, cetyl pyridinium chloride, ergothioneine, and combinations thereof.

1. N-acyl Amino Acid Compound

The compositions of the present invention may comprise an N-acyl amino acid compound. N-acyl amino acid compounds of the present invention correspond to the formula: 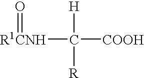
wherein R can be a hydrogen, alkyl (substituted or unsubstituted, branched or straight chain), or a combination of alkyl and aromatic groups. A list of possible side chains of amino acids known in the art are described in Stryer, Biochemistry, 1981, published by W.H. Freeman and Company. R¹ can be C₁ to C₃₀, saturated or unsaturated, straight or branched, substituted or unsubstituted alkyls; substituted or unsubstituted aromatic groups; or mixtures thereof.

Preferably, the N-acyl amino acid compound is selected from the group consisting of N-acyl Phenylalanine, N-acyl Tyrosine, their isomers, their salts, and derivatives thereof. The amino acid can be the D or L isomer or a mixture thereof. N-acyl Phenylalanine corresponds to the following formula: 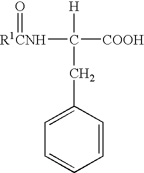
wherein R¹ can be C₁ to C₃₀, saturated or unsaturated, straight or branched, substituted or unsubstituted alkyls; substituted or unsubstituted aromatic groups; or mixtures thereof.

N-acyl Tyrosine corresponds to the following formula: 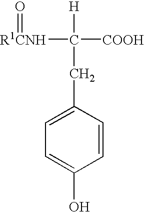
wherein R¹ can be C₁ to C₃₀, saturated or unsaturated, straight or branched, substituted or unsubstituted alkyls; substituted or unsubstituted aromatic groups; or mixtures thereof.

N-undecylenoyl-L-phenylalanine is particularly useful as a topical skin tone evening (lightening or pigmentation reduction) cosmetic agent. This agent belongs to the broad class of N-acyl Phenylalanine derivatives, with its acyl group being a C11 mono-unsaturated fatty acid moiety and the amino acid being the L-isomer of phenylalanine. N-undecylenoyl-L-phenylalanine corresponds to the following formula:

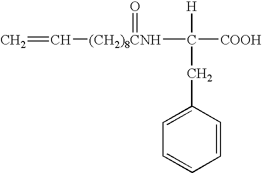

As used herein, N-undecylenoyl-L-phenylalanine is commercially available under the tradename Sepiwhite® from SEPPIC.

When present, the N-acyl amino acid preferably comprises from about 0.0001-25%, more preferably from about 0.001-10%, more preferably from about 0.01-5%, and even more preferably from about 0.02-2.5%, by weight of the composition.

2. Hexamidine

The compositions of the present invention may comprise a hexamidine compound. Hexamidine compounds useful in the present invention correspond to those of the following chemical structure: 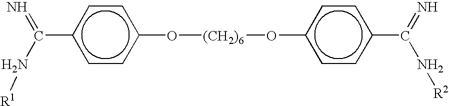
wherein R¹ and R² comprise organic acids (e.g., sulfonic acids, etc.).

Salts and derivatives of hexamidine may also be useful in the present invention. As used herein, hexamidine derivatives include any isomers and tautomers of hexamidine compounds including but not limited to organic acids and mineral acids, for example sulfonic acid, carboxylic acid etc. Preferably, the hexamidine compounds include hexamidine diisethionate, commercially available as Eleastab® HP100 from Laboratoires Serobiologiques.

When present, the hexamidine compound preferably comprises from about 0.0001 to about 25%, more preferably from about 0.001 to about 10%, more preferably from about 0.01 to about 5%, and even more preferably from about 0.02 to about 2.5%,by weight of the composition.

3. Cetyl Pyridinium Chloride

The compositions of the present invention may comprise a safe and effective amount of cetyl pyridinium chloride (CPC). Alternate forms of cetyl pyridinium chloride include those in which one or two of the substitutes on the quaternary nitrogen has a carbon chain length (typically alkyl group) from about 8 to about 20, typically from about 10 to about 18 carbon atoms, while the remaining substitutes (typically alkyl or benzyl group) have a lower number of carbon atoms, such as from about 1 to about 7 carbon atoms (typically methyl or ethyl groups). Dodecyl trimethyl ammonium bromide, tetradecylpyridinium chloride, domiphenbromide, N-tetradecyl-4-ethyl pyridinium chloride, dodecyl dimethyl (2-phenoxyethyl) ammonium bromide, benzyl dimethylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemples of typical quaternary ammonium agents. Other compounds are bis-4-(R-amino)-1-pyridinium alkanes, as disclosed in U.S. Pat. No. 4,206,215.

When present, cetyl pyridinium chloride comprises from about 0.005% to about 10% by weight of the composition, more preferably from about 0.01% to about 5%, more preferably from about 0.05% to about 2%, by weight of the composition.

4. Ergothioneine

The compositions of the present invention may comprise a safe and effective amount of ergothioneine. When present, ergothioneine is included in an amount of from about 0.01% to about 20%, more preferably from about 0.1% to about 15%, even more preferably from about 1% to about 10%, by weight of the composition. A preferred ergothioneine is Thiotaine®, which is a commercial solution of the chemical ergothioneine that is commercially available from Barnet Products.

III. Dermatologically Acceptable Carrier

Compositions of the present invention also comprise a dermatologically acceptable carrier. The carrier is preferably present in an amount from about 50% to about 99.99%, preferably from about 60% to about 99.9%, more preferably from about 70% to about 98%, and even more preferably from about 80% to about 95%, by weight of the composition.

The carrier can be in a wide variety of forms. For example, emulsion carriers, including, but not limited to, oil-in-water, water-in-oil, silicone-in-water, water-in-silicone, water-in-oil-in-water, and oil-in-water-in-silicone emulsions, are useful herein.

Preferred carriers comprise an emulsion such as oil-in-water emulsions and water-in-oil emulsions, e.g., silicone-in-water or water-in-silicone emulsions. As will be understood by the skilled artisan, a given component will distribute primarily into either the water or oil phase, depending on the water solubility/dispensability of the component in the composition. Oil-in-water emulsions are especially preferred.

Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.1% to about 10%, more preferably from about 0.2% to about 5%, of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317 324 (1986).

Suitable emulsions may have a wide range of viscosities, depending on the desired product form. Exemplary low viscosity emulsions, which are preferred, have a viscosity of about 50 centistokes or less, more preferably about 10 centistokes or less, even more preferably about 5 centistokes or less.

The compositions of the present invention can also comprise other topical carriers, and can also comprise oral carriers. For example, another topical carrier can be a surfactant-containing cleanser (e.g., bar, shampoo, foaming cleanser, liquid cleanser, body wash, cleansing cloth, and the like). In such a carrier, the surfactant can be anionic, cationic, zwitterionic, nonionic, or mixtures of these. Another topical carrier example is a color cosmetic (e.g., lipstick, rouge, eye liner, mascara, foundation, nail polish, and the like). An oral carrier can be a beverage, food item, pill, capsule, powder, caplet, and the like.

IV. Optional Components

Compositions of the present invention may contain a wide variety of other components. The optional components, when incorporated into the composition, should be suitable for use in contact with human keratinous tissue without undue toxicity, incompatibility, instability, allergic response, and the like within the scope of sound judgment. The compositions may comprise additional actives, including, but not limited to, hesperedin, mustard seed extract, carnosine, Butylated Hydroxytoluene (BHT) and Butylated Hydroxyanisole (BHA), tetrahydrocurcumin, menthyl anthranilate, vanillin or its derivatives, diethylhexyl syrinylidene malonate, melanostatine, sterol esters; sugar amines, vitamin B3, retinoids, peptides, dialkanoyl hydroxyproline, salicylic acid, phytosterol, sunscreen actives, water soluble vitamins, and oil-soluble vitamins.

The compositions of the present invention may contain a variety of other ingredients that are conventionally used in given product types. The CTFA Cosmetic Ingredient Handbook, Second Edition (1992) describes exemplary cosmetic and pharmaceutical ingredients commonly used in the beauty care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof.

Other optional components useful in compositions of the present invention include those described in U.S. Publication No. 2004-0175347A1, including desquamation actives, such as salicylic acid and zwitterionic surfactants; anti-acne actives, such as resorcinol, sulfur, erythromycin, zinc, dehydroacetic acid; anti-wrinkle actives/anti-atrophy actives; anti-oxidants/radical scavengers, such as tocopherol; chelators, such as furildioxime and derivatives thereof; flavonoids; anti-inflammatory agents; anti-cellulite agents; tanning actives such as dihydroxyacetone; skin lightening agents; antimicrobial and antifungal actives; sunscreen actives; conditioning agents such as glycerol, urea, petrolatum, sucrose polyester, and combinations thereof; thickening agents such as carboxylic acid polymers, crosslinked polyacrylate polymers, polyacrylamide polymers, polysaccharides, gums; and particulate materials.

To minimize complexing, specific types of thickening agents (also known as rheology modifiers) may be employed in compositions containing particular actives or combination of actives. For example, compositions comprising glycyrrhizic acid and/or glycyrrhetinic acid, and an N-acyl amino acid compound preferably employ an anionic or non-ionic thickening agent. Compositions comprising glycyrrhizic acid and/or glycyrrhetinic acid, and a hexamidine compound preferably employ a cationic or non-ionic thickening agent. Compositions comprising glycyrrhizic acid and/or glycyrrhetinic acid, and cetyl pyridinium chloride preferably employ a cationic or non-ionic thickening agent. And compositions comprising glycyrrhizic acid and/or glycyrrhetinic acid, and ergothioneine preferably employ an anionic or non-ionic thickening agent.

### Composition Forms

The topical compositions of the subject invention, including but not limited to lotions, milks, mousses, serums, sprays, aerosols, foams, sticks, pencils, gels, creams and ointments, may comprise a dermatologically acceptable emollient. Such compositions preferably contain from about 2% to about 50% of the emollient. As used herein, “emollient” refers to a material useful for the prevention or relief of dryness, as well as for the protection of the skin. A wide variety of suitable emollients are known and may be used herein. Sagarin, Cosmetics, Science and Technology, 2nd Edition, Vol. 1, pp. 32 43 (1972), contains numerous examples of materials suitable as an emollient. A preferred emollient is glycerin. Glycerin is preferably used in an amount of from about 0.001 to about 20%, more preferably from about 0.01 to about 15%, and even more preferably from about 0.1 to about 10% by weight of the composition.

Compositions of this invention useful for cleansing (“cleansers”) are formulated with a suitable carrier (e.g., as described above, and from about 1% to about 90%, by weight of the composition, of a dermatologically acceptable surfactant).

The physical form of the cleansing compositions is not critical. The compositions can be, for example, formulated as toilet bars, liquids, shampoos, bath gels, hair conditioners, hair tonics, pastes, or mousses. Toilet bars are preferred since this is the form of cleansing agent most commonly used to wash the skin. Rinse-off cleansing compositions, such as shampoos, require a delivery system adequate to deposit sufficient levels of actives on the skin and scalp. A preferred delivery system involves the use of insoluble complexes. For a more complete disclosure of such delivery systems, see, for example, U.S. Pat. No. 4,835,148.

The compositions of the present invention may also be in the form of cosmetics. Suitable cosmetic forms include, but are not limited to, foundations, lipsticks, rouges, mascaras, and the like. Such cosmetic products may include conventional ingredients such as oils, colorants, pigments, emollients, fragrances, waxes, stabilizers, and the like. Exemplary carriers and such other ingredients which are suitable for use herein are described, for example, in U.S. Pat. No. 6,060,547.

### Composition Preparation

Compositions of the present invention are generally prepared by conventional methods such as are known in the art of making topical compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

As noted above, dual compartment packaging may be used, wherein a first personal care composition or composition part is contained within one compartment and a second personal care composition or composition part is contained within the other compartment. The compositions or composition parts are then combined as they are dispensed and applied to keratinous tissue. Separate compositions or composition parts may be contained within single chamber packaging and sequentially or simultaneously applied to an area of keratinous tissue, whereupon the compositions or composition parts are combined. The present invention contemplates methods utilizing these concepts for delivering one or more personal care compositions that include components that have a tendency to complex with one another.

### Methods for Regulating Keratinous Tissue Condition

Compositions of the present invention are useful for regulating a number of mammalian keratinous tissue conditions. Such regulation of keratinous tissue conditions includes prophylactic and therapeutic regulation. Exemplary regulating methods of the present invention are directed to, but are not limited to, thickening keratinous tissue (i.e., building the epidermis and/or dermis and/or subcutaneous layers of the skin and where applicable the keratinous layers of the nail and hair shaft); preventing, retarding, and/or treating uneven skin tone by acting as a lightening or pigmentation reduction cosmetic agent; preventing, retarding, and/or treating atrophy of mammalian skin; softening and/or smoothing lips, hair and nails of a mammal; preventing, retarding, and/or treating itch of mammalian skin; preventing, retarding, and/or treating the appearance of dark under-eye circles and/or puffy eyes; preventing, retarding, and/or treating sallowness of mammalian skin; preventing, retarding, and/or treating sagging (i.e., glycation) of mammalian skin; preventing and/or retarding tanning of mammalian skin; desquamating, exfoliating, and/or increasing turnover in mammalian skin; reducing the size of pores in mammalian skin; regulating oily/shiny appearance of mammalian skin; preventing, retarding, and/or treating hyperpigmentation such as post-inflammatory hyperpigmentation; preventing, retarding, and/or treating the appearance of spider vessels and/or red blotchiness on mammalian skin; preventing, retarding, and/or treating fine lines and wrinkles of mammalian skin; preventing, retarding, and/or treating skin dryness (i.e., roughness, scaling, flaking); and preventing, retarding, and/or treating the appearance of cellulite in mammalian skin.

Regulating keratinous tissue may further include regulating mammalian hair growth, preferably inhibiting and/or retarding hair growth and/or reducing the frequency of shaving. It may also include providing a more noticeable improvement, both tactile and visual, in the appearance and feel of the hair on the skin of a mammal. For example, such regulating methods are directed to making the hair appear softer, finer, and/or less noticeable. Also, such methods may provide ease, frequency, and effectiveness of shaving on a mammal.

Regulating keratinous tissue condition involves topically applying a safe and effective amount of a composition of the present invention to keratinous tissue. The amount of the composition that is applied, the frequency of application and the period of use will vary widely depending upon the level of actives and/or other components of a given composition and the level of regulation desired.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic applications continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions, which are typically applied per application are, in mg composition/cm² skin, from about 0.1 mg/cm² to about 20 mg/cm². A particularly useful application amount is about 0.5 mg/cm² to about 10 mg/cm².

Regulating keratinous tissue condition is preferably practiced by applying a composition in the form of a skin lotion, clear lotion, milky lotion, cream, gel, foam, ointment, paste, emulsion, spray, conditioner, tonic, cosmetic, lipstick, foundation, nail polish, after-shave, or the like, which is intended to be left on the skin or other keratinous tissue for some aesthetic, prophylactic, therapeutic or other benefit (i.e., a “leave-on” composition). After applying the composition to the keratinous tissue (e.g., skin), it is preferably left on for a period of at least about 15 minutes, more preferably at least about 30 minutes, even more preferably at least about 1 hour, even more preferably for at least several hours, e.g., up to about 12 hours. Any part of the external portion of the face, hair, and/or nails can be treated, e.g., face, lips, under-eye area, eyelids, scalp, neck, torso, arms, hands, legs, fingernails, toenails, scalp hair, eyelashes, eyebrows, etc. The application of the present compositions may be done using, e.g., the palms of the hands and/or fingers, an implement; e.g., a cotton ball, swab, pad, etc.

Another approach to ensure a continuous exposure of the keratinous tissue to at least a minimum level of the active is to apply the compound by use of a patch applied, e.g., to the face. Such an approach is particularly useful for problem skin areas needing more intensive treatment (e.g., facial crows feet area, frown lines, under eye area, and the like). The patch can be occlusive, semi-occlusive or non-occlusive. The composition can be contained within the patch or be applied to the skin prior to application of the patch. The patch can also include additional actives such as chemical initiators for exothermic reactions such as those described in PCT application WO 9701313. The patch can also contain a source of electrical energy (e.g., a battery) to, for example, increase delivery of the skin care active and other active agents (e.g., iontophoresis). The patch is preferably left on the keratinous tissue for a period of at least about 5 minutes, more preferably at least about 15 minutes, more preferably still at least about 30 minutes, even more preferably at least about 1 hour, even more preferably at night as a form of night therapy.

Another approach to enhancing the benefits of the actives is use of a kit or regimen of 2 or 3 or 4 or more products and/or treatment procedures (e.g., exfoliation followed by topical treatment with one or more of the actives of the present invention, depilation of hair followed by topical treatment with one or more of the actives of the present invention, and the like). The various components of a regimen can be used in a short period of time (e.g., within an hour), or spread over a longer time frame within a day (e.g., morning and evening), or over even longer time periods (e.g., one step in the regimen done weekly or monthly and the other steps in the regimen done on a more regular basis, e.g., daily). A kit or regimen can also consist of combinations of the carriers noted above, e.g., two or more of a cleanser, a topical leave-on treatment, and an oral supplement.

The present invention also contemplates the delivery of energy, via a device, to keratinous tissue, either simultaneously and/or sequentially with application of the topical compositions. The energy delivery device may deliver energy in a variety of forms, including but not limited to energy in the form of light, heat, sound (including ultrasonic waves), magnetic energy, electromagnetic energy (including radiofrequency waves and microwaves), and combinations thereof. The delivery of energy may be continuous, pulsed, modulated, non-modulated, and combinations thereof. In one embodiment, the energy delivery device is hand-held. Alternatively, the energy delivery device is cordless.

The energy may be applied by holding a device within a single area of keratinous tissue, and subsequently moving the device to another area of tissue (or “stamping”). Alternatively, the energy may be applied as the device is continuously moved, or scanned, across the surface of the tissue. The device may be held in substantially continuous contact with the surface of the keratinous tissue, as with laser devices, or may be held at a short distance from the keratinous tissue with the energy directed toward the surface, as with flash lamps.

A temperature change may be simultaneously induced in the keratinous tissue or alternatively, in a compound applied to the surface of the tissue. This temperature change is in addition to any temperature change induced by the delivered energy itself. For example, the keratinous tissue may be slightly warmed prior to delivery of energy, or alternatively, the keratinous tissue may be cooled after delivery of energy.

For energy derived from ultraviolet light sources, the wavelength will generally fall within the UV-A range, from about 315-400 nm, where “nm” means 1×10-9 meters. For energy derived from visible light sources, the wavelength will generally range from about 400 nm to about 700 nm. For energy derived from infrared (IR) light sources, the wavelength will generally range from about 700 run to about to about 3000 nm. The amount of energy delivered, or “output fluence,” may be in the range of about 1 J/cm² to about 100 J/cm², where “J” means Joules. For pulsed light sources, the pulse length may range from about 0.001 seconds to about 3 seconds, with an average pulse duration of from about 0.001 seconds to about 1 second. The surface area of keratinous tissue to be covered will vary depending on the application. These and other parameters relevant to delivery of energy depend upon the type of treatment and the type of tissue to be treated, and will appropriately be selected by one of skill in the art.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

Content in formulation (g component per 100 g formulation)

| Component | A | B | C | D | E | F |
|---|---|---|---|---|---|---|
| Disodium EDTA | 0.100 | 0.100 | 0.100 | 0.100 | 0.100 | 0.100 |
| Carnosine | 5.000 | 0 | 0 | 0 | 0 | 0 |
| Vanillin acetate | 0 | 2.000 | 0 | 0 | 0 | 0 |
| Tetrahydrocurcumin | 0 | 0 | 0.500 | 0 | 0 | 0 |
| Glycyrrhetinic acid | 0 | 0 | 0 | 0.300 | 0 | 0 |
| Thiotaine ® | 0 | 0 | 0 | 0 | 5.000 | 0 |
| Sinablanca ® | 0 | 0 | 0 | 0 | 0 | 2.500 |
| Niacinamide | 4.000 | 4.000 | 4.000 | 4.000 | 4.000 | 4.000 |
| Citric acid | 1.500 | 0 | 0 | 0 | 0 | 0 |
| Isohexadecane | 3.000 | 3.000 | 0 | 0 | 3.000 | 3.000 |
| Isopropyl isostearate | 1.330 | 1.330 | 0 | 0 | 1.330 | 1.330 |
| Isopropyl N-laurosylsarcosinate | 0 | 0 | 5.000 | 6.000 | 0 | 0 |
| Sucrose polycottonseedate | 0.670 | 0.670 | 0 | 0 | 0.670 | 0.670 |
| Polymethylsilsesquioxane | 0.250 | 0.250 | 0.250 | 0.250 | 0.250 | 0.250 |
| Cetearyl glucoside + cetearyl alcohol | 0.200 | 0.200 | 0.200 | 0.200 | 0.200 | 0.200 |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 isoparaffin + laureth-7 | 3.000 | 2.000 | 2.000 | 2.000 | 2.000 | 2.000 |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to | to | to | to | to | to |
| | 100 | 100 | 100 | 100 | 100 | 100 |
| TOTAL | 100 | 100 | 100 | 100 | 100 | 100 |

Content in formulation (g component per 100 g formulation)

| Component | G | H | I | J | K | L |
|---|---|---|---|---|---|---|
| Disodium EDTA | 0.100 | 0.100 | 0.100 | 0.100 | 0.100 | 0.100 |
| Hesperidin | 1.000 | 0 | 0 | 0 | 0 | 0 |
| Glycyrrhizic acid, ammonium salt | 0 | 0.300 | 0 | 0 | 0 | 0 |
| Cetyl pyridinium chloride | 0 | 0 | 0.200 | 0 | 0 | 0 |
| Menthyl aanthranilate | 0 | 0 | 0 | 5.000 | 0 | 0 |
| Oxynex ® | 0 | 0 | 0 | 0 | 2.000 | 0 |
| Butylated hydroxyl toluene | 0 | 0 | 0 | 0 | 0 | 1.000 |
| Niacinamide | 4.000 | 4.000 | 4.000 | 4.000 | 4.000 | 4.000 |
| Polyquaternium 37 | 0 | 0 | 1.500 | 0 | 0 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl alcohol | 0.200 | 0.200 | 0.200 | 0.200 | 0.200 | 0.200 |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 isoparaffin + laureth-7 | 2.000 | 2.000 | 0 | 2.000 | 2.000 | 2.000 |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to | to | to | to | to | to |
| | 100 | 100 | 100 | 100 | 100 | 100 |
| TOTAL | 100 | 100 | 100 | 100 | 100 | 100 |

| Component | M | N |
|---|---|---|
| Disodium EDTA | 0.1 | 0.1 |
| Niacinamide | 4.0 | 4.0 |
| Isohexadecane | 0 | 3.0 |
| Isopropyl isostearate | 0 | 1.33 |
| Isopropyl N-laurosylsarcosinate | 6.0 | 0 |
| Sucrose polycottonseedate | 0.67 | 0.67 |
| Polymethylsilsesquioxane | 0.25 | 0.25 |
| Cetearyl glucoside + cetearyl alcohol | 0.2 | 0.2 |
| Behenyl alcohol | 0.4 | 0.4 |
| Ethylparaben | 0.2 | 0.2 |
| Propylparaben | 0.1 | 0.1 |
| Cetyl alcohol | 0.32 | 0.32 |
| Stearyl alcohol | 0.48 | 0.48 |
| Tocopheryl acetate | 0.5 | 0.5 |
| PEG-100 stearate | 0.1 | 0.1 |
| Glycerin | 7.0 | 7.0 |
| Titanium dioxide | 0.604 | 0.604 |
| Polyacrylamide + C13-14 isoparaffin + laureth-7 | 2.0 | 2.0 |
| Panthenol | 1.0 | 1.0 |
| Benzyl alcohol | 0.4 | 0.4 |
| Dimethicone + dimethiconol | 2.0 | 2.0 |
| Water (to 100 g) | to 100 | to 100 |
| TOTAL | 100 | 100 |

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition, comprising:
a) a safe and effective amount of glycyrrhizic acid and/or glycyrrhetinic acid;
b) a safe and effective amount of a first active selected from the group consisting of N-acyl amino acid compounds, hexamidine, cetyl pyridinium chloride, ergothioneine, and combinations thereof; and
c) a dermatologically acceptable carrier.

2. The personal care composition of claim 1, wherein the first active comprises an N-acyl amino compound.

3. The personal care composition of claim 1, wherein the first active comprises hexamidine.

4. The personal care composition of claim 1, wherein the first active comprises cetyl pyridinium chloride.

5. The personal care composition of claim 1, wherein the first active comprises ergothioneine.

6. The personal care composition of claim 2, wherein the first active further comprises ergothioneine.

7. The personal care composition of claim 3, wherein the first active further comprises an N-acyl amino compound.

8. The personal care composition of claim 3, wherein the first active further comprises cetyl pyridinium chloride.

9. The personal care composition of claim 7, wherein the first active further comprises cetyl pyridinium chloride.

10. The personal care composition of claim 9, wherein the first active further comprises ergothioneine.

11. The personal care composition of claim 4, wherein the first active further comprises ergothioneine.

12. The personal care composition of claim 2, further comprising an anionic or non-ionic thickening agent.

13. The personal care composition of claim 3, further comprising a non-ionic or cationic thickening agent.

14. The personal care composition of claim 4, further comprising a non-ionic or cationic thickening agent.

15. The personal care composition of claim 5, further comprising an anionic or non-ionic thickening agent.

16. The personal care composition of claim 1, further comprising a safe and effective amount of a second active selected from the group consisting of sugar amines, vitamin B₃, retinoids, peptides, dialkanyl hydoxyproline, salicylic acid, phytosterol, sunscreen actives, water soluble vitamins, oil-soluble vitamins, their derivatives, their precursors, and combinations thereof.

17. A personal care composition, comprising:
a) a safe and effective amount of glycyrrhizic acid and/or glycyrrhetinic acid;
b) a safe and effective amount of niacinamide;
c) a safe and effective amount of vitamin E or a derivative thereof;
d) a safe and effective amount of panthenol;
e) a safe and effective amount of a sunscreen agent; and
f) a dermatologically acceptable carrier.

18. The personal care composition of claim 17, wherein the vitamin E or a derivative thereof comprises tocopheryl acetate.

19. A method of regulating the condition of mammalian keratinous tissue, comprising the steps of:
a) combining a first personal care composition or composition part comprising a first active selected from the group consisting of glycyrrhizic acid, glycyrrhetinic acid, N-acyl amino acid compounds, ergothioneine, and combinations thereof; with a second personal care composition or composition part comprising a second active selected from the group consisting of hexamidine, cetyl pyridinium chloride, and combinations thereof; and
b) applying the first personal care composition or composition part and the second personal care composition or composition part to the mammalian keratinous tissue,
wherein step a) is conducted directly before and/or during step b).
